(19) United States
(12) Patent Application Publication
(10) Pub. No.: US 2013/0071432 A1
(43) Pub. Date: Mar. 21, 2013

(54) COMBINATION VIROTHERAPY FOR CANCER

(71) Applicants: Robert Silverman, Beachwood, OH (US); Babal Kant Jha, Cleveland, OH (US)

(72) Inventors: Robert Silverman, Beachwood, OH (US); Babal Kant Jha, Cleveland, OH (US)

(73) Assignee: THE CLEVELAND CLINIC FOUNDATION, Cleveland, OH (US)

(21) Appl. No.: 13/621,959

(22) Filed: Sep. 18, 2012

Related U.S. Application Data
(60) Provisional application No. 61/536,838, filed on Sep. 20, 2011.

(51) Int. Cl. A61K 39/12; A61P 37/02; A61P 31/12; A61K 39/205; A61P 35/00

(52) U.S. Cl. 424/205.1; 424/204.1; 424/224.1

(57) ABSTRACT

One aspect of the present disclosure relates to a method for treating a subject with cancer. The method includes administering an oncolytic virus simultaneously, sequentially, or separately in combination with an immunomodulatory agent in an amount effective to suppress both antiviral immunity and angiogenesis associated with the cancer.

### RELATED APPLICATION

This application claims the benefit of U.S. Provisional Patent Application Ser. No. 61/536,838, filed Sep. 20, 2011, the entirety of which is hereby incorporated by reference for all purposes.

### TECHNICAL FIELD

The present disclosure relates generally to combination virotherapy for cancer and, more particularly, to the combined use of an oncolytic virus and an immunomodulatory agent for treating cancer.

### BACKGROUND

Cancer is diagnosed in more than 1 million people every year in the United States (U.S.) alone. In spite of numerous advances in medical research, cancer remains the second leading cause of death in the U.S. In industrialized nations, roughly one in five persons will die of cancer. In the search for novel strategies, oncolytic virus therapy has recently emerged as a viable approach to specifically kill tumor cells. Unlike conventional gene therapy, it uses replication competent viruses that are able to spread through tumor tissue by virtue of viral replication and concomitant cell lysis, providing an alternative treatment for cancer. Viruses have now been engineered to selectively replicate and kill cancer cells.

Oncolytic viruses may utilize multiple mechanisms of action to kill cancer cells—cell lysis, cell apoptosis, anti-angiogenesis and cell necrosis. The virus infects the tumor cell and then begins to replicate. The virus continues to replicate until the host cell's membrane lyses (bursts) as the tumor cell can no longer contain the virus. The tumor cell is destroyed and the newly created viruses are spread to neighboring cancer cells to continue the cycle. Oncolytic viruses are intended to replicate only in cancer cells and to pass through normal tissue without causing harm. Hence, once all the tumor cells are eradicated, the oncolytic virus no longer has the ability to replicate and the immune system clears it from the body.

Over the past few years, new insights into the molecular mechanisms of viral cytotoxicity have provided the scientific rationale to design more effective oncolytic viruses. Recent advances in molecular biology have allowed the design of several genetically modified viruses, such as adenovirus and herpes simplex virus that specifically replicate in, and kill, tumor cells. On the other hand, viruses with intrinsic oncolytic capacity are also being evaluated for therapeutic purposes. Although the efficacy of oncolytic virus therapy in general has been demonstrated in preclinical studies, the therapeutic efficacy in clinical trials is still not optimal.

### SUMMARY

One aspect of the present disclosure relates to a method for treating a subject with cancer. The method includes administering an oncolytic virus simultaneously, sequentially, or separately in combination with an immunomodulatory agent in an amount effective to suppress both antiviral immunity and angiogenesis associated with the cancer in the subject.

Another aspect of the present disclosure relates to a method for treating or ameliorating a solid tumor in a subject. The method includes administering to the subject an effective amount of an oncolytic virus to the solid tumor. The oncolytic virus is capable of selectively killing the cells of the solid tumor. The method also includes administering to the subject an immunomodulatory agent in an amount effective to suppress both angiogenesis associated with the solid tumor and innate immunity to the oncolytic virus.

Another aspect of the present disclosure relates to a method for modulating sensitivity of a cancer cell to infection by an oncolytic virus. The method includes contacting the cancer cell with the oncolytic virus. The method also includes introducing into the subject an immunomodulatory agent in an amount effective to modulate antiviral immunity and sensitize the cancer cell to cytolytic activity of the oncolytic virus.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIGS. 1A-E show that sunitinib inhibits RNase L and protein kinase R(PKR) in a cell-free system and in intact cells. FIG. 1A shows inhibition of RNase L and RNase LNA335 by sunitinib as measured by FRET. FIG. 1B shows that autophosphorylation of purified PKR in response to poly(rI):poly(rC) is inhibited by sunitinib. FIG. 1C shows cells (as indicated) incubated with or without sunitinib (5 μg/ml) for 2 h and then mock transfected or transfected with poly(rI):poly(rC) [p(rI):p(rC)] (2 μg/ml) for 3 hrs prior to isolating RNA and analyzing rRNA on RNA chips. FIG. 1D shows inhibition of PKR phosphorylation of eIF2α by sunitinib (5 μM) in different cell lines (indicated) as monitored in Western blots. Levels of total eIF2α, PKR and RNase L are also shown. FIG. 1E shows cell lines (as indicated) treated with 5 μM sunitinib for 2 h followed by vesicular stomatitis virus (VSV) infection for 8 h. Cell survival was determined by MTS assay and results presented as percent of control. Results are an average of two experiments done in triplicate (Su, sunitinib;

FIGS. 2A-D show antitumor efficacy of sunitinib combined with VSV. Prostate cancer cells, PC3 (10⁶ cells in media plus matrigel) were implanted s.c. in athymic nude (nu/nu) mice (FIG. 2A). Sunitinib treatments (oral 20 mg/kg daily) started on day 16 post-implantation. Intratumoral infections of VSV (10⁶ pfu/mouse) were done on the days indicated (arrows). Tumor sizes were measured using a digital caliper. Viral titers from PC3 tumors 3 days after infection with VSV alone, or treated with sunitinib and also infected with VSV (FIG. 2B). Wilcoxon signed rank test (p=0.004) was calculated using Prizm software. Human renal carcinoma cells ACHN (2.5×10⁶ cell in media plus matrigel) were implanted s.c. into nude mice and treated with sunitinib and/or VSV as indicated (FIG. 2C). Mouse breast cancer cell line 4T1 (10⁴ cells in PBS plus matrigel) was implanted s.c. into immunocompetent BALB-cJ mice (FIG. 2D). Sunitinib treatment and VSV inoculation into tumors are indicated. Data analysis was done with Prizm software and represent mean±s.e.m (NT, no treatment; Su, sunitinib);

FIG. 3 is a schematic illustration showing that sunitinib inhibits intracellular innate immunity by blocking viral infection of RNase L (panel A) and PKR (panel B);

FIGS. 4A-D are a series of schematic illustrations comparing effects of cyclophosphamide (CPA) (FIG. 4A) and sunitinib (FIG. 4B) on antiviral antibody titers and oncolytic virotherapy in C56/b16 mice (FIGS. 4C-D) (i.t., intratumoral; i.v., intravenous);

FIGS. 5A-B are a series of schematic illustrations showing enhanced extravasation of VSV in nude mouse models of castration-resistant prostate cancer (*, times of treatments for the orthotopic implanted mice dependent on kinetics of metastatic spread);

FIGS. 6A-C show sunitinib inhibits RNase L and PKR in a cell-free system and in intact cells. Levels of total PKR, RNase L, and β-actin in Western blots probed with specific antibodies are shown (FIG. 6A). Cells (as indicated) were incubated with or without sunitinib (5 μg/ml) for 2 h and then mock transfected or transfected with poly(rI):poly(rC) [p(rI):p(rC)] (2 μg/ml) for 3 hrs prior to isolating RNA and analyzing rRNA on RNA chips (FIG. 6B). Cell lines (as indicated) were treated with 5 μM sunitinib for 2 h followed by VSV infection for 8 h (FIG. 6C). Cell survival was determined by MTS assay and results presented as percent of control. Results are an average of two experiments done in triplicate (Su, sunitinib); and

FIG. 7 is a graph showing that sunitinib/VSV combination selectively kills cancer cells in culture. The indicated cancer and normal cells were treated with 5 μM sunitinib (Su) for 2 h followed by VSV infection for 8 h. Cell survival was determined by MTS assay and results presented as percent of control. Results are an average of two experiments done in triplicate.

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the present disclosure pertains. Commonly understood definitions of molecular biology terms can be found in, for example, Rieger et al., Glossary of Genetics: Classical and Molecular, 5th Ed., Springer-Verlag: New York, 1991, and Lewin, Genes V, Oxford University Press: New York, 1994. The definitions provided herein are to facilitate understanding of certain terms used frequently herein and are not meant to limit the scope of the present disclosure.

In the context of the present disclosure, the terms “neoplastic cell,” “tumor cell,” or “cancer cell” can refer to a cell that proliferates at an abnormally high rate. A new growth comprising neoplastic cells is a neoplasm, also known as a “tumor.” A tumor is an abnormal tissue growth, generally forming a distinct mass that grows by cellular proliferation more rapidly than normal tissue growth. A tumor may show a partial or total lack of structural organization and functional coordination with normal tissue. A tumor can encompass hematopoietic tumors as well as solid tumors. A tumor may be benign (benign tumor) or malignant (malignant tumor or cancer). Malignant tumors can be broadly classified into three major types: malignant tumors arising from epithelial structures are called carcinomas; malignant tumors that originate from connective tissues, such as muscle, cartilage, fat, or bone are called sarcomas; and malignant tumors affecting hematopoietic structures (structures pertaining to the formation of blood cells) including components of the immune system are called leukemias and lymphomas.

As used herein, the term “oncolytic virus” can refer to a virus that preferentially replicates in, and kills, neoplastic cells. An oncolytic virus may be a naturally-occurring (or native) virus or an engineered virus. Oncolytic viruses can also encompass immunoprotected and reassortant viruses. An oncolytic virus may be “naturally-occurring” (or “native”) when it can be isolated from a source in nature and has not been intentionally modified by humans (e.g., in the laboratory). For example, an oncolytic virus can be from a “field source”; that is, from an infected animal. An oncolytic virus may be “engineered” when it has been modified (e.g., genetically-modified) by human intervention.

As used herein, the term “infection by oncolytic virus” can refer to the entry and replication of an oncolytic virus in a cell. Similarly, “infection of a tumor cell by an oncolytic virus” can refer to the entry and replication of an oncolytic virus in the cells of the tumor.

As used herein, the term “subject” can include a vertebrate, such as a mammal (e.g., a human, horse pig, rabbit, dog, sheep, goat, non-human primate, cow, cat, guinea pig or rodent), a fish, a bird or a reptile or an amphibian. The term does not denote a particular age or sex. Thus, adult and newborn subjects, as well as fetuses, whether male or female, are intended to be encompassed by the term. The terms “patient” or “subject” may be used interchangeably herein, and can refer to a subject afflicted with a disease or disorder, such as cancer.

As used herein, the term “sensitivity” of a cancer or tumor cell to an oncolytic virus can refer to the susceptibility of the cancer or tumor cell to the lytic effect of the oncolytic virus. For example, sensitivity of a cancer or tumor cell to an oncolytic virus may be indicated by increased lysis of the cancer or tumor cell as compared to a non-tumor or non-cancer cell (e.g., a normal cell). The sensitivity of a tumor or cancer cell may also be demonstrated by a reduction of the symptom(s) caused by the tumor or cancer cell in a subject.

As used herein, the term “administration” of an oncolytic virus and/or an immunomodulatory agent to a subject can refer to the act of administering the oncolytic virus and/or the immunomodulatory agent to the subject in a manner so that the oncolytic virus and/or the immunomodulatory agent can contact a target neoplastic cell (or cells). The route by which the oncolytic virus and/or the immunomodulatory agent is administered, as well as the formulation, carrier or vehicle, will depend on the location as well as the type of the target neoplastic cell(s). As discussed in further detail below, a wide variety of administration routes and formulations can be employed.

As used herein, the terms “contacted” or “exposed”, when applied to a cell, can be used interchangeably to describe the process by which an oncolytic virus and an immunomodulatory agent are delivered to a target neoplastic cell (or cells) or are placed in direct juxtaposition with the target neoplastic cell(s). To achieve cell killing, both agents (e.g., the oncolytic virus and the immunomodulatory agent) can be delivered to a neoplastic cell in a combined amount effective to kill the neoplastic cell, i.e., to induce programmed cell death or apoptosis.

As used herein, the terms “killing”, “programmed cell death” and “apoptosis” can be used interchangeably to describe a series of intracellular events that lead to target neoplastic cell death.

As used herein, the term “effective amount” can refer to an amount of an oncolytic virus and/or an immunomodulatory agent sufficient to result in an intended effect. For a combination oncolytic virus and immunomodulatory agent used to treat or ameliorate a tumor, an effective amount can include an amount of the oncolytic virus and an amount of the immunomodulatory agent sufficient to alleviate or eliminate the symptoms of the tumor, or to slow down the progress of the tumor.

As used herein, the terms “treatment”, “treat,” “treating” or “ameliorating” can refer to reducing the effects of a disease (e.g., cancer), condition, or symptom of the disease or condition. Thus, in some instances, treatment can refer to a 10%, 20%, 30%, 40%, 50%, 60%, 70%, 80%, 90% or 100% reduction or amelioration in the severity of a disease, condition or symptom of the disease or condition. For example, cancer may be considered to be treated if there is a 10% reduction in one or more symptoms of cancer in a subject (as compared to control). Thus, the reduction can be a 10%, 20%, 30%, 40%, 50%, 60%, 70%, 80%, 90%, 100% or any percent reduction in between 10% and 100% as compared to native or control levels. It will be understood that treatment does not necessarily refer to a cure or complete ablation of the disease, condition or symptoms of the disease or condition.

As used herein, the term “angiogenesis” can refer to a process of tissue vascularization that involves the development of new vessels. Angiogenesis may occur via one of three mechanisms: (1) neovascularization, where endothelial cells migrate out of pre-existing vessels beginning the formation of the new vessels; (2) vasculogenesis, where the vessels arise from precursor cells de novo; or (3) vascular expansion, where existing small vessels enlarge in diameter to form larger vessels.

As used herein, the term “plaque-forming unit” (pfu) can mean one infectious virus particle.

As used herein, the term “potentiate” can mean additive or even synergistic increase in the level of cell killing above that seen for one treatment modality alone.

As used herein, the term “combined amount effective to kill a neoplastic cell” can refer to that the amount of an immunomodulatory agent and an oncolytic virus sufficient so that, when combined within the neoplastic cell, cell death is induced. The combined effective amount of the agents (e.g., the immunomodulatory agent and the oncolytic virus) can be an amount that induces more cell death than the use of either agent alone.

As used herein, the term “inhibitor” can mean either that the given compound or agent is capable of inhibiting the activity of a respective protein or other substance in a cell (e.g., a neoplastic cell) at least to a certain amount. This can be achieved either by a direct interaction of the compound or agent with the given protein or substance (“direct inhibition”), or by an interaction of the compound or agent with other proteins or other substances in or outside the cell, which leads to an at least partial inhibition of the activity of the protein or substance (“indirect inhibition”).

As used herein, the terms “suppress” or “suppresses” can refer to a complete or partial reduction in a specified activity (e.g., viral-induced cell lysis), level (e.g., intra- or extracellular protein concentration) or intended effect (e.g., a symptom associated with cancer).

As used herein, the term “antiviral immunity” can refer to innate and adaptive immune responses associated with viral infection.

As used herein, the term “synergistic” can indicate that a therapeutic effect is greater than would have been expected based on adding the effects of each agent (e.g., an oncolytic virus and an immunomodulatory agent) applied as a monotherapy.

### Overview

Oncolytic viruses are therapeutically used microbes (either naturally occurring or genetically-engineered) that preferentially infect and replicate in cancer cells with the goal of eliminating malignant tumors without serious toxicity. Oncolytic viruses target tumor cells precisely because the same genetic alterations that allow malignant tumor cells to proliferate and survive also promote growth of lytic viruses. Oncolytic viruses have a significant advantage compared with chemotherapy in that oncolytic viruses selectively eliminate cancer cells, thereby sparing non-cancerous tissues. Additionally, drug-resistant cancer cells and cancer stem cells retain their susceptibility to oncolytic viruses, and oncolytic viruses are also effective in hypoxic environments characteristic of solid tumors. Despite their early promise, however, oncolytic therapy has not reached its potential against cancer, due principally to the host immune response. To be effective, oncolytic viruses must spread, infect, and lyse every cancer cell or cancer stem cell without first being inhibited and then cleared by the innate and adaptive immune responses (respectively). Thus, pre-existing immunity or immunity as a result of repeated administration of an oncolytic virus reduces the effectiveness of therapy.

Experiments were performed to investigate whether the balance between viral spread and cell lysis and antiviral immune responses can be shifted to favor viral replication and spread by suppressing immune responses in the tumor. In particular, combination therapy using sunitinib and vesicular stomatitis virus (VSV) on cell death was investigated in the following intact cancer cells: human prostate cancer (DU145, PC3 and LNCaP); human kidney cancer (ACHN, 786-0 and Renca); mouse breast cancer (4T1); and human brain cancer (U251 and SNB19). Cell viability was measured by MTS assays. In all of the cancer cells, there was a minimal reduction in cell viability by sunitinib or VSV alone, whereas the combination of sunitinib and VSV greatly enhanced cell death. For example, sunitinib or VSV alone had partial effects against PC3 tumors, whereas a dramatic and persistent anti-tumor effect of the combined therapy with sunitinib and VSV was observed against PC3 tumors. To extend these findings to another tumor type, ACHN human renal cell carcinoma cells were implanted into nude mice. It was subsequently found that direct inoculation of an attenuated VSV strain into the ACHN tumors or orally delivered sunitinib alone retarded tumor growth, whereas the combined treatment caused complete regression of the ACHN tumors. Identical findings were also observed in BALBc mice implanted with mouse 4T1 breast cancer cells and treated with wild-type VSV and sunitinib. Finally, it was also observed that there was no apparent toxicity associated with sunitinib/VSV therapy and, after cessation of therapy, there was no recurrence of cancer.

Thus, the present disclosure is based, at least in part, on the discovery that the combined synergistic effects of sunitinib and an oncolytic virus can eliminate cancer cells directly by enhancing the viral cytopathic effect in tumors while indirectly suppressing tumor growth by inhibiting angiogenesis. The two principal pathways for viral resistance are the 2′,5′-oligoadenylate synthetase-RNase L pathway that degrades viral RNA, and the RNA-dependent protein kinase (PKR) pathway, which inhibits viral protein synthesis. Sunitinib is a kinase inhibitor of vascular endothelial growth factor receptor (VEGF-R) and platelet-derived growth factor receptor (PDGF-R), which is used clinically to block angiogenesis. Sunitinib can also impair antiviral innate immune responses by blocking the activities of RNase L and PKR. Without wishing to be bound by theory, it is believed that infection of tumor cells with an oncolytic virus, such as VSV results in activation of both RNase L and PKR, and that treatment with an immunomodulatory agent, such as sunitinib inhibits angiogenesis and intracellular innate immunity by preventing viral activation of RNase L and PKR. Based at least in part on the foregoing, the present disclosure provides a combination virotherapy for treating cancer, as well as a method for potentiating or modulating sensitivity of cancer cells to infection by an oncolytic virus.

The ability of an oncolytic virus to mediate tumor destruction depends on the ability of the virus to replicate throughout the tumor mass. Antiviral immunity in the tumor mass abrogates the effectiveness of oncolytic virotherapy by inhibiting the spread of the virus within the tumor. Advantageously, the present disclosure can shift the balance between (1) viral spread and cell lysis and (2) antiviral immune responses to favor viral replication and spread by suppressing immune responses (e.g., innate immunity and/or adaptive immunity) in the tumor, as well as suppressing angiogenesis associated with the tumor.

Accordingly, one aspect of the present disclosure can include a method for treating or ameliorating cancer (e.g., a solid tumor) in a subject, comprising administering an oncolytic virus in combination with an immunomodulatory agent in an amount effective to suppress both antiviral immunity and angiogenesis associated with the cancer. The step of administering an oncolytic virus can be done simultaneously, sequentially, or separately in combination with administration of an immunomodulatory agent. In some instances, for example, the oncolytic virus can be administered before or concurrent with the immunomodulatory agent. In other instances, the oncolytic virus can be administered following administration of the immunomodulatory agent to the subject. The step of administering an oncolytic virus in combination with an immunomodulatory agent can be repeated as desired to ameliorate or treat cancer in the subject.

### Cancers

In another aspect, cancers treatable by the present disclosure can include any type of cancer. In some instances, a cancer treatable by the present disclosure can include a condition characterized by abnormal cell growth. In other instances, a cancer treatable the present disclosure can include a condition characterized by a significant loss of control of cell proliferation. Cancers treatable by the present disclosure can include metastatic as well as non-metastatic cancer or tumors. Cancers treatable by the present disclosure can further include benign tumors, as well as cell masses that include dysplastic or hyperplastic cells.

A cancer may be diagnosed using criteria generally accepted in the art, including the presence of a malignant tumor.

Types of cancer that may be treated according to the present disclosure can include, but are not limited to, hematopoietic cell cancers including leukemias and lymphomas, colon cancer, lung cancer, kidney cancer, pancreas cancer, endometrial cancer, thyroid cancer, oral cancer, ovarian cancer, laryngeal cancer, hepatocellular cancer, bile duct cancer, squamous cell carcinoma, prostate cancer, breast cancer, cervical cancer, colorectal cancer, melanomas and any other tumors. Solid tumors, such as sarcomas and carcinomas can include, but are not limited to, fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, and CNS tumors (e.g., a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma).

In one example, a cancer treatable by the present disclosure can include prostate cancer, such as castration-resistant prostate cancer.

In another example, a cancer treatable by the present disclosure can include kidney cancer, such as clear-cell renal cell carcinoma.

In another example, a cancer treatable by the present disclosure can include breast cancer.

### Oncolytic Viruses

Oncolytic viruses that can be used to practice the present disclosure can include, but are not limited to, oncolytic viruses that are a member in the family of myoviridae, siphoviridae, podpviridae, teciviridae, corticoviridae, plasmaviridae, lipothrixviridae, fuselloviridae, poxyiridae, iridoviridae, phycodnaviridae, baculoviridae, herpesviridae, adnoviridae, papovaviridae, polydnaviridae, inoviridae, microviridae, geminiviridae, circoviridae, parvoviridae, hepadnaviridae, retroviridae, cycloviridae, reoviridae, birnaviridae, paramyxoviridae, rhabdoviridae, fioviridae, orthomyxoviridae, bunyaviridae, arenaviridae, leviviridae, picornaviridae, sequiviridae, comoviridae, potyviridae, caliciviridae, astroviridae, nodaviridae, tetraviridae, tombusviridae, coronaviridae, glaviviridae, togaviridae, and barnaviridae. Immunoprotected or reassortant viruses of other oncolytic viruses are also encompassed by the present disclosure. Furthermore, a combination of at least two oncolytic viruses can also be employed to practice the present disclosure.

The oncolytic virus used to practice the present disclosure may be naturally-occurring or modified (e.g., chemically-treated and/or genetically-modified). In some instances, the oncolytic virus may be chemically or biochemically pretreated (e.g., by treatment with a protease, such as chymotrypsin or trypsin) prior to administration to the subject. Pretreatment with a protease can remove the outer coat or capsid of the virus, and may increase the infectivity of the virus. In other instances, the oncolytic virus may be coated in a liposome or micelle to reduce or prevent an immune response. For example, the virus may be treated with chymotrypsin in the presence of micelle-forming concentrations of alkyl sulfate detergents to generate a new infectious subviral particle. The oncolytic virus may also be a reassortant virus or an intermediate sub-viral particle. In other instances, it may be preferable that the oncolytic virus is not a vehicle for delivering a gene for the purpose of gene therapy. For example, viruses have been engineered to deliver the adenoviral E1A gene, the p53 tumor suppressor gene, prodrug-encoding genes or genes under a radiation-inducible promoter. In some cases, these viruses may not replicate preferentially in neoplastic cells.

The oncolytic virus may be a recombinant oncolytic virus resulting from the recombination/reassortment of genomic segments from two or more genetically distinct oncolytic viruses. Recombination/reassortment of oncolytic virus genomic segments may occur in nature following infection of a host organism with at least two genetically distinct oncolytic virus. Recombinant virions can also be generated in cell culture, for example, by co-infection of permissive host cells with genetically distinct oncolytic viruses. In some instances, a recombinant oncolytic virus resulting from reassortment of genome segments from two or more genetically distinct oncolytic viruses can be used. In other instances, a recombinant oncolytic virus that has undergone recombination in the presence of chemical mutagens (e.g., dimethyl sulfate and ethidium bromide) or physical mutagens (e.g., ultraviolet light and other forms of radiation) can be used to practice the present disclosure. In further instances, the present disclosure can include the use of recombinant oncolytic viruses that: comprise deletions or duplications in one or more genome segments; comprise additional genetic information as a result of recombination with a host cell genome; or comprise synthetic genes, such as genes encoding agents that suppress antiviral immune responses.

The oncolytic virus may be modified by incorporation of mutated coat proteins, such as into the virion outer capsid. The proteins may be mutated by replacement, insertion or deletion. Replacement can include the insertion of different amino acids in place of the native amino acids. Insertions can include the insertion of additional amino acid residues into the protein at one or more locations. Deletions can include deletions of one or more amino acid residues in the protein. Such mutations may be generated by methods known in the art. For example, oligonucleotide site-directed mutagenesis of the gene encoding for one of the coat proteins may result in the generation of the desired mutant coat protein. Expression of the mutated protein in oncolytic virus-infected mammalian cells in vitro can result in the incorporation of the mutated protein into the oncolytic virion particle.

The oncolytic virus may be modified to express one or more targeting or therapeutic proteins, separately or as a part of other expressed proteins. Generally, a virus can be engineered to include one or more additional genes that target the virus to cells of interest (see, e.g., U.S. Pat. No. 7,429,481). In some instances, expression of the gene results in expression of a ligand on the surface of the virus containing one or more domains that bind to antigens, ligands or receptors that are specific to tumor cells, or are upregulated in tumor cells compared to normal tissue. Appropriate targeting ligands will depend on the cell or cancer of interest and will be known to those skilled in the art. For example, a virus can be engineered to bind to antigens, ligands or receptors that are specific to tumor cells or tumor-associated neovasculature, or are upregulated in tumor cells or tumor-associated neovasculature compared to normal tissue. Other examples of tumor antigens are disclosed in U.S. Patent Publication No. 2010/0172877 A1.

In one example, the oncolytic virus can include a native or modified VSV. VSV is a negative RNA stranded rhabdovirus, which is pathogenic for horses, cattle and swine. In contrast, VSV infections are usually asymptomatic in humans. Natural infections with VSV are extremely rare in humans, and therefore so are pre-existing antibodies against VSV. Advantageously, VSV has broad tissue tropism (i.e., it will infect all types of tumor cells). Additionally, while VSV infects and kills many types of tumor cells, viral growth is attenuated in normal cells.

### Immunomodulatory Agents

In another aspect, an immunomodulatory agent used in combination with an oncolytic virus can include any compound, molecule, or substance capable of suppressing both antiviral immunity and angiogenesis associated with a tumor or cancer. In some instances, the immunomodulatory agent is capable of suppressing innate immunity and/or adaptive immunity to the oncolytic virus. Innate immunity associated with viral infection includes various non-specific mechanisms, such as stimulation of interferon (IFN) (e.g., Type I IFN) production and enhancement of the ability of natural killer cells to lyse virally-infected cells. The Type I IFN response is normally beneficial to the host but, in the case of therapy with an oncolytic virus, the opposite is true because the Type I IFN system restricts the spread of the oncolytic virus to every tumor cell. Once a virus infects a tumor cell, the RNase L and PKR pathways (discussed above) are activated and thereby limit virus replication and virus-induced lytic cell death (respectively). Advantageously, the immunomodulatory agent can suppress or inhibit the activities of RNase L and PKR in tumor cells infected with an oncolytic virus. By suppressing the activities of these proteins, viral replication and protein synthesis can occur without disruption, thereby exerting a direct tumor cytopathic effect. Thus, in some instances, the immunomodulatory agent can potentiate or sensitize a cancer cell to infection by an oncolytic virus.

The ability of the immunomodulatory agent to also suppress angiogenesis provides a further mechanism for treating cancer. It is known that angiogenesis performs a critical role in the development of cancer. Solid tumors smaller than 1 to 2 cubic millimeters are not vascularized. To spread, they need to be supplied by blood vessels that bring oxygen and nutrients and remove metabolic wastes. Beyond the critical volume of 2 cubic millimeters, oxygen and nutrients have difficulty diffusing to the cells in the center of the tumor, causing a state of cellular hypoxia that marks the onset of tumoral angiogenesis. New blood vessel development is an important process in tumor progression. It favors the transition from hyperplasia to neoplasia, i.e., the passage from a state of cellular multiplication to a state of uncontrolled proliferation characteristic of tumor cells. Neovascularization also influences the dissemination of cancer cells throughout the entire body, eventually leading to metastasis formation. Thus, the combined synergistic effects of the immunomodulatory agent and the oncolytic virus can ameliorate or treat cancer by directly enhancing the viral cytopathic effect in tumors, while indirectly suppressing tumor growth by suppressing (or inhibiting) angiogenesis associated with the tumor.

In another aspect, an immunomodulatory agent can include a compound, molecule, or substance capable of both suppressing innate immunity in a tumor cell and suppressing (e.g., inhibiting) angiogenesis associated with a cancer. In some instances, the immunomodulatory agent may be capable of: (1) suppressing or inhibiting the activity of RNase L and PKR in a tumor cell infected with an oncolytic virus; and (2) inhibiting VEGF-R and PDGF-R. In one example, the immunomodulatory agent can include sunitinib. Unlike sunitinib, other anti-angiogenesis agents that have been used to enhance the efficacy of oncolytic viruses (e.g., bevacizumab), thrombospondin 1 peptide, trichostatin A and cilengtide) do not impair innate immunity to the viruses. Additionally, the use of sunitinib as part of a combination therapy with an oncolytic virus differs fundamentally from sunitinib's original clinical purpose, i.e., inhibiting angiogenesis.

In another example, an immunomodulatory agent can include a flavonol.

In another aspect, the immunomodulatory agent is capable of suppressing innate adaptive immunity to the oncolytic virus. The adaptive (or humoral/cell-mediated) immune system provides specific effector mechanisms in antiviral immunity. One such mechanism includes antibodies, such as neutralizing antibodies that bind to the virus (usually to the viral envelope or capsid proteins) and block the virus from binding and gaining entry to the host cell. Virus-specific antibodies may also act as opsonins in enhancing phagocytosis of virus particles. In addition, viral proteins expressed on the surface of a host cell may serve as targets for virus-specific antibodies, and may lead to complement-mediated lysis of the infected cell or may direct a subset of natural killer cells to lyse the infected cell (via antibody-directed cellular cytotoxicity). Another mechanism of the adaptive immune response to viral infection includes the action of cytotoxic T cells (e.g., CD8+ cytotoxic T lymphocytes), which are involved in clearing established viral infections.

Thus, in some instances, an immunomodulatory agent can include any compound, molecule, or substance capable of suppressing an antiviral antibody response in a cell infected with an oncolytic virus. In one example, the immunomodulatory agent can include an alkylating agent, such as cyclophosphamide (CPA). CPA has been used as a chemotherapeutic drug against cancer, as an immunosuppressant for treating autoimmune diseases, and for use in solid organ transplantation and in clinical trials with oncolytic viruses. CPA can suppress the primary and anamnestic antiviral antibody response during oncolytic virotherapy using, for example, VSV.

### Other Agents

In another aspect, a vascular agent capable of enhancing or increasing vascular permeability can be administered to the subject in combination with the immunomodulatory agent and the oncolytic virus. In some instances, the vascular agent can be administered to the subject prior to administration of the immunomodulatory agent and the oncolytic virus. For example, the vascular agent can be administered about 1 day (e.g., less than, equal to, or greater than 1 day) prior to administering the immunomodulatory agent and the oncolytic virus. In other instances, the vascular agent can be administered concurrent with, or subsequent to, administration of the immunomodulatory agent and the oncolytic virus to the subject.

The vascular agent can include any compound, molecule, or substance capable of enhancing or increasing vascular permeability in the subject. In one example, the vascular agent can specifically enhance or increase vascular permeability in a tumor (e.g., a solid tumor). By enhancing or increasing vascular permeability, administration of the vascular agent can promote or increase delivery of an oncolytic virus to a cancer or tumor cell. In some instances, the vascular agent can comprise a cytokine. Examples of cytokines capable of increasing vascular permeability are known in the art. In one example, the vascular agent can include tumor necrosis factor-alpha (TNF-α). In another example, the vascular agent can include interleukin-2 (IL-2). Where the agent includes IL-2, it will be appreciated that IL-2 can be administered to the subject in a non-toxic amount.

### Formulations and Routes of Administration

In another aspect, where clinical applications of the present disclosure are contemplated, it will be necessary to prepare pharmaceutical compositions comprising a combination of an immunomodulatory agent, an oncolytic virus and, optionally, a vascular agent in a form appropriate for the intended application.

A “combination” of an immunomodulatory agent, an oncolytic virus and, optionally, a vascular agent may be formulated together in the same dosage form or may be formulated in separate dosage forms. Separate dosage forms may be the same form or different forms, for administration by the same mode, or by different modes of administration. Furthermore, administration of a combination of an immunomodulatory agent and an oncolytic virus (and optionally a vascular agent), when not together in the same dosage form, can mean that the immunomodulatory agent and the oncolytic virus (and optionally the vascular agent) are administered concurrently to the subject being treated, and may be administered at the same time or sequentially in any order or at different points in time. Thus, the immunomodulatory agent and the oncolytic virus (and optionally the vascular agent) may be administered separately but sufficiently close in time so as to provide the desired therapeutic effect.

The effective amount to be administered to a subject can vary depending on many factors, such as the pharmacodynamic properties of the oncolytic virus and the immunomodulatory agent (and optionally the vascular agent), the modes of administration, the age, health and weight of the subject, the nature and extent of the disease state, the frequency of the treatment, and the type of concurrent treatment, if any, and the virulence and titre of the oncolytic virus. For example, one of skill in the art can determine the appropriate amount of the oncolytic virus for administration based on the above factors. The oncolytic virus may be administered initially in a suitable amount that may be adjusted as required, depending on the clinical response of the subject. The effective amount of the oncolytic virus can be determined empirically and can depend on the maximal amount of the virus that can be administered safely, and the minimal amount of the virus that produces the desired result.

The oncolytic virus may be administered to the subject using standard methods of administration. In one example, the oncolytic virus can be administered systemically. In another example, the oncolytic virus can be administered by injection at the disease site. Where the particular disease state is a solid tumor, for example, the oncolytic virus can be administered by injection at the tumor site. In other instances, the oncolytic virus may be administered orally or parenterally, or by any other standard method known in the art.

The concentration of the oncolytic virus to be administered will vary depending on the virulence of the particular viral strain to be administered and on the nature of the neoplastic cells that are being targeted. In one example, a dose of less than about 10⁹ pfu can be administered to a human subject. In another example, between about 10² to about 10⁹ pfu, between about 10² to about 10⁷ pfu, between about 10³ to about 10⁶ pfu, or between about 10⁴ to about 10⁵ pfu may be administered in a single dose.

One of skill in the art can also determine, using the above factors, the appropriate amount of the immunomodulatory agent (and optionally the vascular agent) to administer to a subject. The effective amount of the immunomodulatory agent, for example, can be determined empirically and will depend on the amount and strain of oncolytic virus being administered, the maximum amount of the immunomodulatory agent that can be safely administered, and the minimal amount of the immunomodulatory agent that can be administered to potentiate or sensitize a target neoplastic cell to the oncolytic virus.

The immunomodulatory agent (and optionally the vascular agent) may be administered to the subject using standard methods of administration. In one example, the immunomodulatory agent can be administered systemically. In another example, the immunomodulatory agent can be administered by injection at the disease site. Where the disease state is a solid tumor, for example, the immunomodulatory agent can be administered by injection at the tumor site. In other examples, the immunomodulatory agent may be administered orally or parenterally, or by any other standard method known in the art.

The total amount of the immunomodulatory agent (and optionally the vascular agent) may be administered in a single dose or in multiple doses spread out over 1 day or several days. The frequency and duration of administration of doses can be readily determined. The schedule of dosing will depend, at least in part, on the length of time that the oncolytic virus is to be administered. For example, the immunomodulatory agent (and optionally the vascular agent) may be administered once to a subject, or may be administered 2 to 4 times per day.

Effective amounts of a combination of oncolytic virus and immunomodulatory agent (and optionally a vascular agent) can be given repeatedly, depending upon the effect of the initial treatment regimen. Administrations can be given periodically, while monitoring any response. It will be recognized by a skilled person that lower or higher dosages may be given according to the administration schedules and routes selected.

To aid in administration, the oncolytic virus, in combination together with the immunomodulatory agent (and optionally the vascular agent), may be formulated as an ingredient in a pharmaceutical composition. Therefore, in some instances, there is provided a pharmaceutical composition comprising an oncolytic virus, an immunomodulatory agent, an optional agent capable of increasing vascular permeability, and a pharmaceutically acceptable diluent. In other instances, there is provided a pharmaceutical composition comprising an oncolytic virus, an optional vascular agent, and a pharmaceutically acceptable diluent. In further instances, there is provided a pharmaceutical composition comprising an immunomodulatory agent, an optional vascular agent, and a pharmaceutically acceptable diluent. In still further instances, there is provided a vascular agent and a pharmaceutically acceptable diluents. The compositions may routinely contain pharmaceutically acceptable concentrations of salt, buffering agents, preservatives and various compatible carriers.

The proportion and identity of the pharmaceutically acceptable diluent can be determined by the chosen route of administration, compatibility with a live virus and, where applicable, compatibility with the chemical stability of the immunomodulatory agent (and optionally the vascular agent), and standard pharmaceutical practice. Generally, pharmaceutical compositions can be formulated with components that will not significantly impair the biological properties of the oncolytic virus, or cause degradation of or reduce the stability or efficacy of the immunomodulatory agent.

Pharmaceutical compositions can be prepared by known methods for the preparation of pharmaceutically acceptable compositions suitable for administration to subjects, such that an effective quantity of an active substance (or substances) is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the pharmaceutical compositions can include, albeit not exclusively, solutions of the oncolytic virus, optionally with the immunomodulatory agent, in association with one or more pharmaceutically acceptable vehicles or diluents, and contained in buffer solutions with a suitable pH and iso-osmotic with physiological fluids.

Pharmaceutical compositions may be administered to a subject in a variety of forms depending on the selected route of administration, as will be understood by those skilled in the art. In one example, pharmaceutical compositions of the present disclosure may be administered orally or parenterally. Parenteral administration can includes intravenous, intraperitoneal, subcutaneous, intramuscular, transepithelial, nasal, intrapulmonary, intrathecal, rectal and topical modes of administration. Parenteral administration may be by continuous infusion over a selected period of time. In another example, pharmaceutical compositions can be directly administered to a disease site, e.g., via direct intratumoral injection.

Pharmaceutical compositions can be administered orally, for example, with an inert diluent or with an assimilable carrier, enclosed in hard or soft shell gelatin capsules, or compressed into tablets. For oral therapeutic administration, the oncolytic virus may be incorporated, optionally together with an immunomodulatory agent, with an excipient and be used in the form of ingestible tablets, buccal tablets, troches, capsules, elixirs, suspensions, syrups, wafers, and the like.

Solutions of oncolytic virus, optionally together with an immunomodulatory agent (and optionally the vascular agent), may be prepared in a physiologically suitable buffer. Under ordinary conditions of storage and use, these preparations contain a preservative to prevent the growth of microorganisms, but that will not inactivate the live virus. A person skilled in the art would know how to prepare suitable formulations. Conventional procedures and ingredients for the selection and preparation of suitable formulations are described, for example, in Remington's Pharmaceutical Sciences and in The United States Pharmacopeia: The National Formulary (USP 24 NF19) published in 1999.

The dose of the pharmaceutical composition that is to be used can depend on the particular cancer being treated, the severity of the cancer, the individual subject parameters including age, physical condition, size and weight, the duration of the treatment, the nature of concurrent therapy (if any), the specific route of administration, and other similar factors that are within the knowledge and expertise of the health practitioner. These factors are known to those of skill in the art and can be addressed with minimal routine experimentation.

It will be appreciated that the oncolytic virus, optionally in combination with an immunomodulatory agent and/or vascular agent, or pharmaceutical compositions comprising the oncolytic virus and an immunomodulatory agent (and optionally the vascular agent), either together in the same formulation or different formulations, may also be packaged as a kit, containing instructions for use of the oncolytic virus and the immunomodulatory agent (and optionally the vascular agent), including the use of the oncolytic virus, or use of the oncolytic virus in combination with the immunomodulatory agent (and optionally the vascular agent), to ameliorate or treat cancer.

The following examples are for the purpose of illustration only and are not intended to limit the scope of the claims, which are appended hereto.

### Example 1

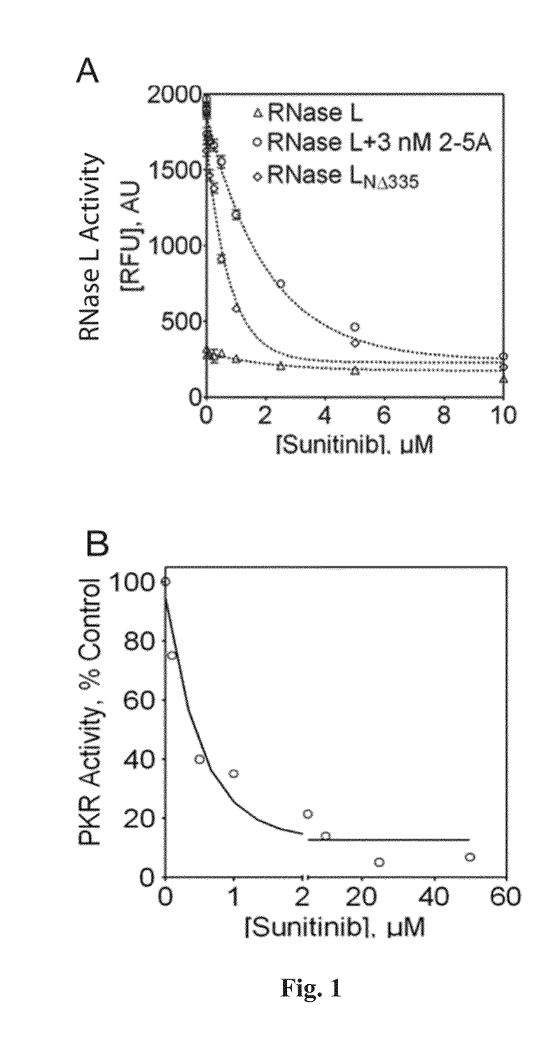

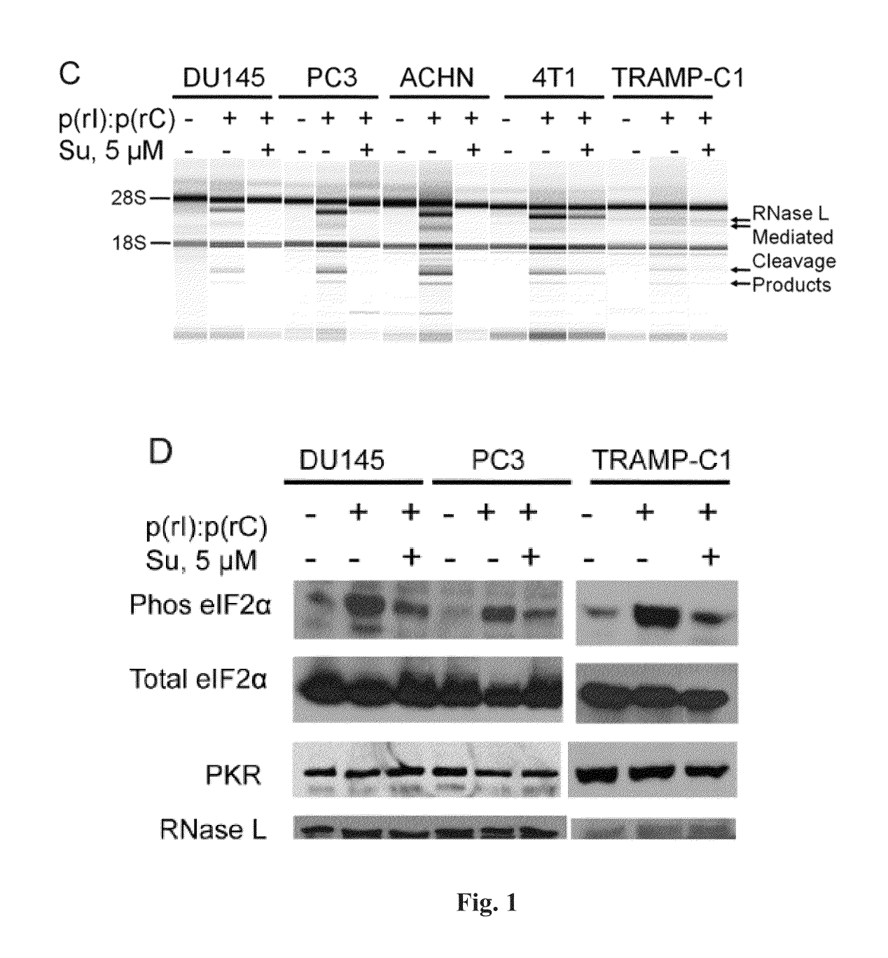

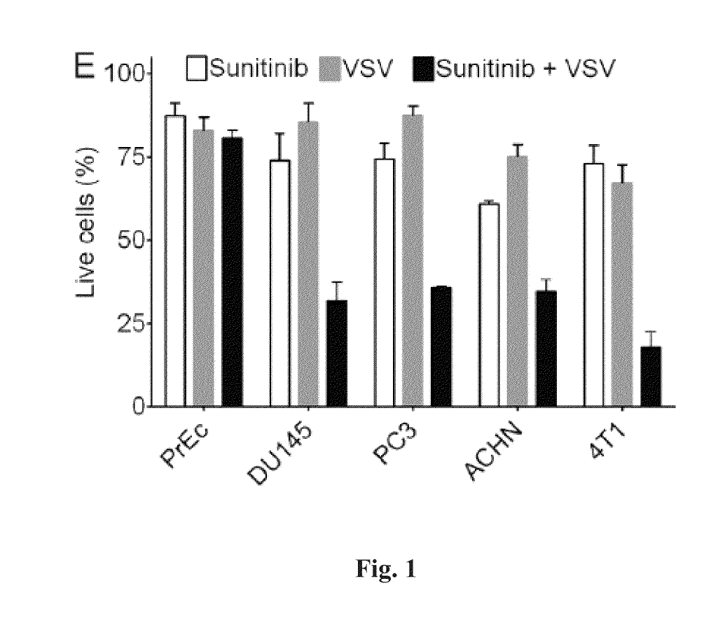

Previously, we reported potent inhibition of both RNase L and PKR by sunitinib in cell-free systems, in cell culture, and in mice. Dose response curves for inhibition of RNase L and PKR by sunitinib in vitro from that study are shown (FIGS. 1A-B). Either full length RNase L activated by 2-5A, or a constitutively active, truncated mutant RNase L(NΔ335), were inhibited by suntinib at a IC₅₀ (concentration required to inhibit enzyme activity by 50%) of 1.4 μM as determined in fluorescence resonance energy transfer (FRET) assays for RNase L activity (FIG. 1A). Similarly, sunitinib inhibited PKR activity with an IC₅₀ of 0.3 μM as determined in PKR autophosphorylation assays in the presence of the activator synthetic dsRNA, poly(rI):poly(rC) (FIG. 1B). To determine the effect of sunitinib on RNase L activity in intact cancer cells, human prostate cancer (DU145, PC3), renal cell carcinoma (ACHN) and mouse breast cancer (4T1) cell lines were transfected with poly(rI):poly(rC) for 3 hrs to activate OAS and RNase L, total RNA was isolated and separated on RNA chips (FIG. 1C). RNase L cleaves rRNA in intact ribosomes producing a unique and characteristic set of discrete RNA cleavage products. RNase L cleavage of rRNA in human cells (DU145, PC3, ACHN and 4T1) gives a slightly different pattern of rRNA cleavage products than mouse cells (TRAMP-C1). As expected, RNase L activity was observed in the poly(rI):poly(rC)-transfected cancer cells, whereas there was little or no rRNA cleavage products in the poly(rI):poly(rC)-transfected and sunitinib treated cells. Therefore, these cells have an intact OAS-RNase L pathway and sunitinib treatment effectively blocked RNase L activation in all of the cancer cell lines.

The effect of sunitinib treatments (5 μM) on PKR activity in response to poly(rI):poly(rC) transfection of DU145, PC3 and TRAMP-C1 cells was monitored in Western blots (FIG. 1D). Sunitinib effectively inhibited PKR as determined by reduced phosphorylation of its substrate, eIF2α. To determine the effects of suntinib and oncolytic virus (OV) against prostate cancer cells, we used one of the most frequently studied OVs, the negative RNA stranded rhabdovirus, VSV (Indiana strain). The effect of sunitinib and VSV on survival of prostate cancer (PC3, DU145), renal cell carcinoma (ACHN), breast cancer (4T1) cell lines and on primary prostatic epithelial cells (PrEC) was determined (FIG. 1E). Cells were pre-incubated in the absence or presence of sunitinib (5 μM) for 2 h and were either left uninfected or were infected with VSV at a multiplicity of infection (MOI) of 1.0 for 8 h (a single cycle of virus replication). Cell viability was measured by MTS assays using the CellTiter96® Aqueous kit (Promega Inc.). There was a minimal (typically about 25%) reduction in cell viability by sunitnib or VSV alone. Interestingly, the combination of sunitinib and VSV greatly enhanced cell death in all of the cancer cells (70% to 80%), whereas primary prostatic epithelial cells (PrEC) were resistant to the combination treatment. PC3 cells, which are normally resistant to VSV, presumably due to the presence of both the OAS/RNase L and PKR systems, were rendered susceptible to VSV by sunitinib treatment. These cell culture studies suggested that sunitinib renders cancer cells more susceptible to the lytic effect of VSV infections by impairing innate immunity, an effect that is independent of angiogenesis.

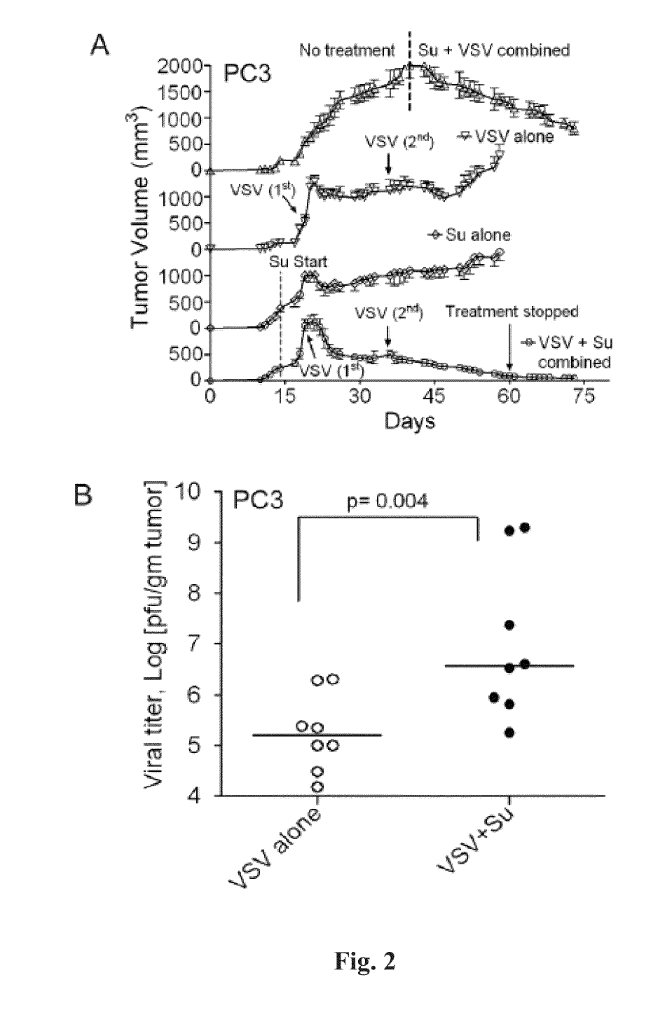

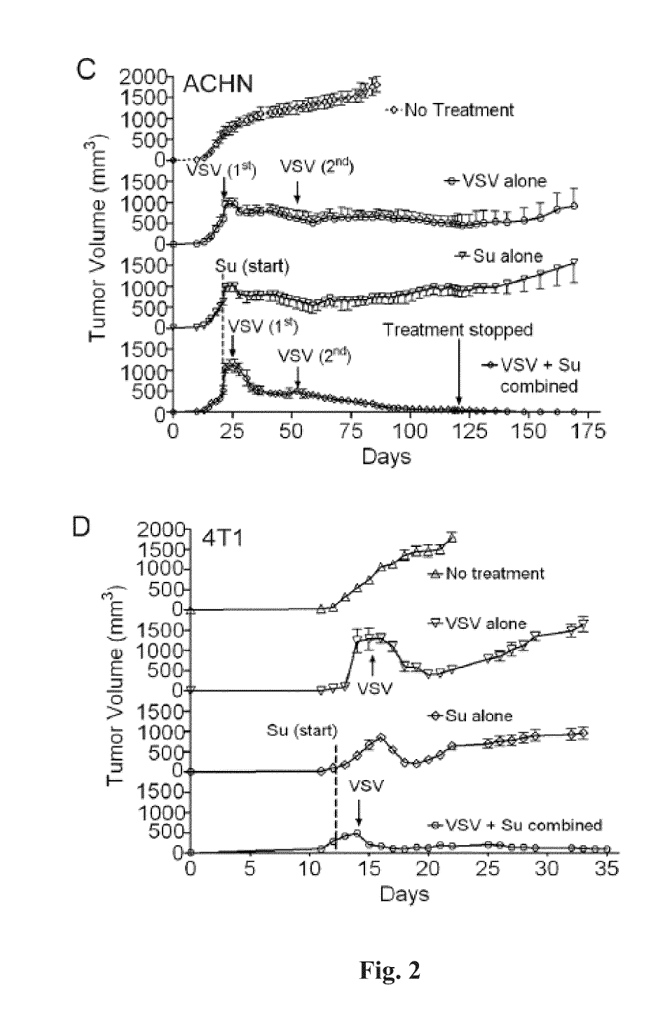

PC3 was chosen for tumor studies in mice because these cells express both RNase L and PKR (FIG. 1D) and are relatively resistant to the oncolytic activity of VSV. In addition, PC3 cells originated from advanced, metastatic (bone) prostate cancer and are castration resistant. To determine the effects of sunitinib and VSV on prostate tumors, PC3 cells in matrigel were implanted subcutaneously (s.c.) in nude mice. Once PC3 tumor sizes reached 6-8 mm in diameter (12 days), mice were separated into four groups (n=8): no treatment; sunitinib alone (by oral gavage at 20 mg/kg daily beginning on day 16 post-implantation); VSV infection alone [10⁶ plaque forming units (pfu)/mouse injected intratumorally (i.t.) on days 18 & 35 post-implantation], and combined sunitinib treatment with VSV infection (FIG. 2A). The dose of suntinib used in all of our studies, 20 mg/kg, results in serum concentrations that are equivalent to those of patients receiving suntinib.

Suntinib or VSV alone had partial effects against PC3 tumors, reducing tumor growth rates but not causing tumors to completely regress. In contrast, there was a dramatic and persistent anti-tumor effect of the combined therapy with VSV and sunitinib. When tumors in the untreated mice approached the size requiring euthanasia, the mice received daily treatments with sunitinib orally and the tumors were inoculated with VSV. The combined treatment also caused regression of late-stage tumors (FIG. 2A, top plot to the right of the vertical dashed line). In mice receiving the combined treatment beginning from day 18, the tumors did not return after the treatments ceased on day 60 (FIG. 2A, bottom plot, see arrow labeled “treatment stopped”). To demonstrate the role of innate immunity in the combined treatment, we measured viral titers in the tumors after 3 days of treatment with VSV alone or with VSV plus sunitinib (FIG. 2B). The combination therapy enhanced median viral yields in the tumors by 23-fold, thereby confirming that sunitinib impaired innate immunity in vivo.

To extend these findings to another tumor type, ACHN human renal cell carcinoma cells were implanted s.c. into nude mice (FIG. 2C). An attenuated VSV strain (M51R), which triggers a higher interferon response than wild type VSV and may have an improved safety profile compared with wild type VSV, was used. (However, neither wild type VSV nor mutant VSV M51R showed any apparent toxicity in any of our mouse experiments). Direct inoculation of VSVM51R into the ACHN tumors or orally delivered sunitinib alone retarded tumor growth, whereas the combined treatment caused complete regression of the ACHN tumors (FIG. 2C). The mice were considered cured because the tumors did not reappear, even 6 weeks after sunitinib treatment ceased. In addition, we have observed identical findings in immunocompetent BALBc mice implanted with mouse 4T1 breast cancer cells treated with wild type VSV and sunitinib (FIG. 2D).

### Example 2

To determine effects of sunitinib on innate immunity pathways in vivo, activation of both RNase L and PKR is monitored in tumors implanted s.c. in mice. Sunitinib is obtained from the Cleveland Clinic pharmacy and dissolved in PBS at 4 mg/ml. PC3 cells (2×10⁶ in media plus matrigel, 1:1, in a final volume of 150 μl) are implanted s.c. into a single flank of athymic nude homozygous (nu/nu) NCR mice (Taconic, obtained through the Case Comprehensive Cancer Center, Cleveland). Tumor sizes are measured daily with a digital caliper. When the tumors reach 6-8 mm in diameter (typically 12 to 13 days), mice are divided into four groups (n=5): no treatment; sunitinib alone (20 mg/kg daily by oral gavage); VSV intratumoral (i.t.) inoculation alone [10⁶ plaque forming units (pfu)/mouse] two days later; and VSV i.t. inoculation two days later in mice also receiving sunitinib.

At 24, 48 and 72 hrs post-infection mice are euthanized, blood collected, and the tumors excised. Tumors are immediately cut each into three parts with a scalpel and individual sections placed in dry ice, RNAlater (Qiagen), and formalin. Tumor tissue frozen in dry ice is thawed in PBS and homogenized using a Sample Grinding kit including a silica beads and a pestle (GE Healthcare). After centrifuging, supernatants are collected and used for performing viral plaque assays on the indicator mouse cell line, L929. Viral plaque assays are also performed on sera collected at the time of euthanasia. The supernatants of the tissue homogenates are used for Western blot assays.

Total and phosphorylated levels of PKR and its substrate, eIF2α, are measured in Western blots by probing with specific antibodies (Cell Signaling, Inc.). RNase L activity is monitored in tumors by examining the integrity of rRNA in tumor samples preserved in RNAlater. During viral infections, RNase L activation leads to the cleavage of both 28S and 18S at unique sites, as we showed previously (FIG. 1C). To identify RNase L mediated cleavage products in rRNA, the RNA is isolated using TRIzol (LifeTechnologies) and separated in RNA chips on an Agilent BioAnalyzer 2100. In addition, viral growth in tumors is assessed in fixed tissue sections. Formalin fixed and paraffin embeded (FFPE) tissues are sectioned and stained with antibody to VSV G protein (KeraFAST, Inc.).

### Example 3

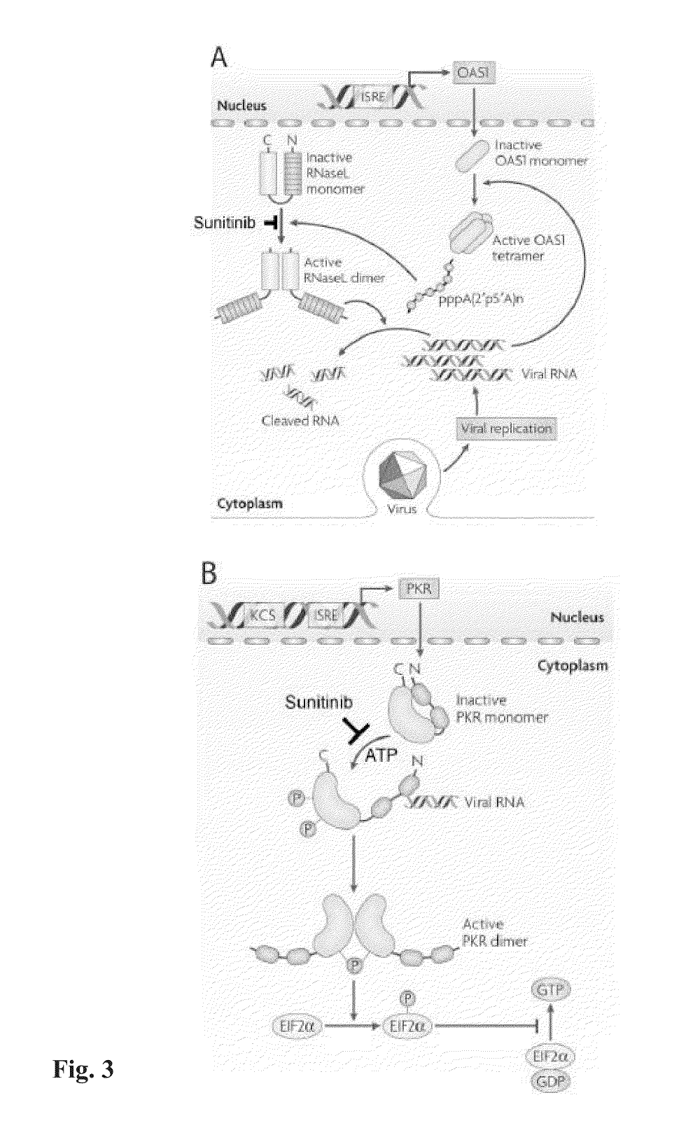

To determine how sunitinib enhances oncolytic virotherapy in vivo, the contributions of RNase L and PKR are established, individually and together, on tumor growth. Previously, it was reported that a vaccinia virus encoded protein, E3L, inhibits both RNase L and PKR by sequestering dsRNA, an activator of OAS (and subsequently RNase L) and PKR (FIG. 3). E3L in PC3 is stably expressed in cells in order to simultaneous inhibit RNase L and PKR. The E3L cDNA (purchased as a synthetic cDNA from GenScript) is expressed as an N-terminal flag-tag protein in mammalian expression vector pCMV-ENTRY (Origene). PC3 cells are transfected with the E3L construct and selected for stable expression with G418. E3L expression is monitored in Western blots with antibody against the flag-tag. To demonstrate that E3L is functional, the PC3 cells expressing E3L, or containing empty vector as a control, are transfected with poly(rI):poly(rC) (1 μg/ml) for 3 hrs. RNase L activation is monitored by rRNA cleavages in RNA chips, whereas PKR activation is monitored in Western blots probed with antibody against phosphorylated and unphosphorylated PKR and eiF2α. nt PC3/E3L and PC3/control cells are then implanted into the flanks of nude mice and proceed with the tumor growth experiments as described above with four groups of mice (n=8): no treatment; VSV alone; sunitinib (daily) alone; and VSV plus sunitinib (daily). By comparing tumor growth rates between the vector control and E3L expressing PC3 cells, the contributions of RNase L and PKR (together) on tumor regression in response to VSV and sunitinib are established.

Recently, we reported that the ns2 gene of the coronavirus, mouse hepatitis virus (MHV), encodes a protein that shuts down the OAS/RNase L pathway. Ns2 is a 2′,5′-phosphodiesterase that degrades 2-5A, the activator of RNase L, and thus prevents RNA cleavage by this enzyme. To selectively block the OAS-RNase L system, the ns2 protein from a cDNA is stably expressed in PC3 cells. Expression is confirmed in Western blots and then it is determined if RNase L activity is blocked by transfecting with poly(rI):poly(rC) prior to examining rRNA in RNA chips. Subsequently, the PC3 cells expressing MHV ns2 are implanted s.c. into nude mice and VSV/sunitinib treatments are performed. By monitoring tumor growth, the contribution of RNase L by itself to the antitumor effects of VSV in the presence and absence of sunitinib against PC3 tumors in mice is determined.

To determine the relative contribution of both RNase L and PKR, RNase L and PKR levels in PC3 cells with lentiviruses encoding different shRNAs against mRNAs for RNase L or PKR are separately down-regulated. RNase L and PKR levels are monitored in Western Blots probed with a monoclonal antibody against human RNase L that we prepared, and antibody against PKR (from Cell Signaling, Inc.). Subsequently, the cells are transfected with poly(rI):poly(rC) prior to monitoring rRNA cleavage in RNA chips and phosphorylated and unphosphorylated PKR and eIF2α in Western blots. PC3 cells are generated that stably express the most effective shRNA against either RNase L or PKR. Effects of suntinib and VSV on cell survival are determined in PC3 cells in culture by MTS assays and then against s.c. implanted tumors in nude mice treated with sunitinib and VSV alone and in combination.

### Example 4

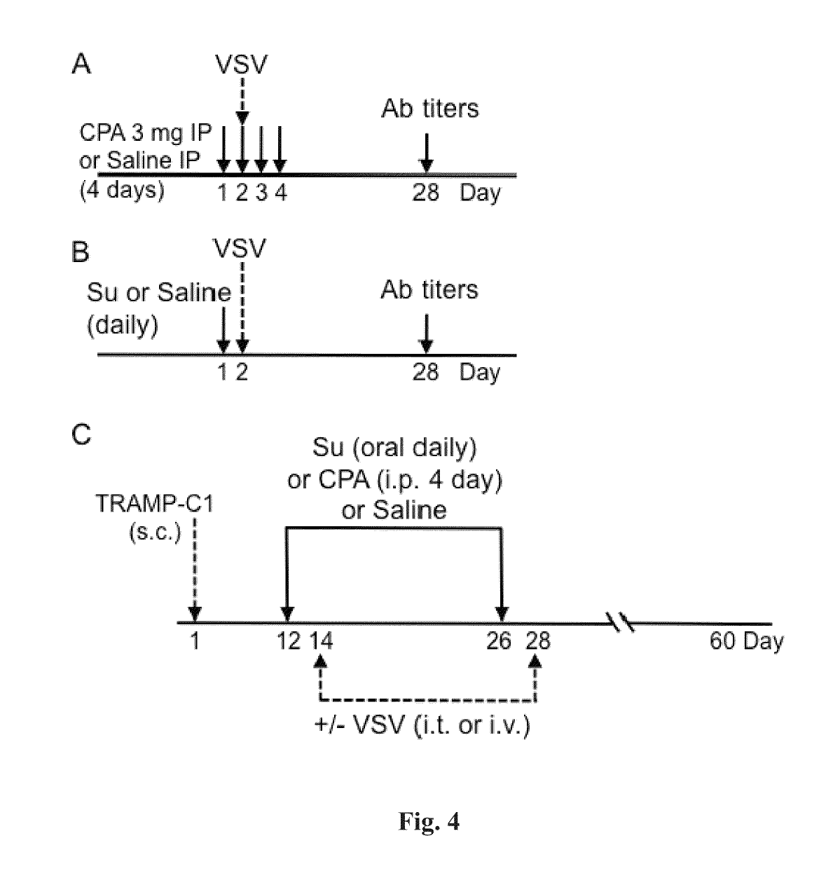

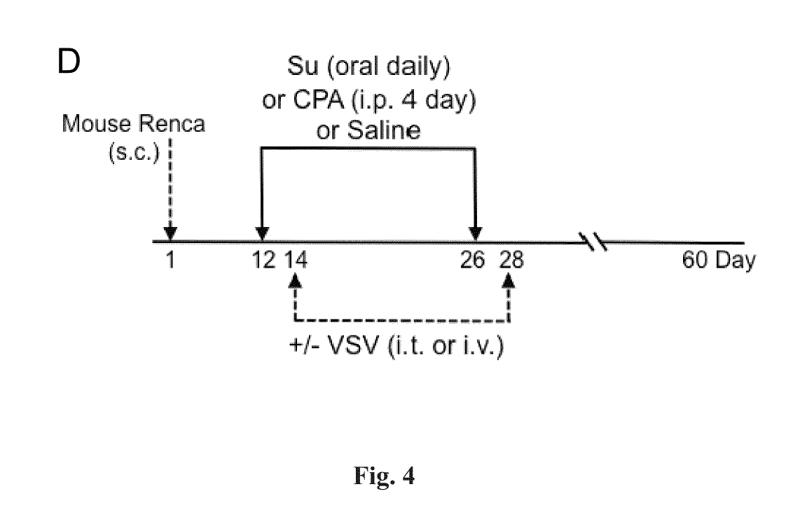

The effects of an immunosuppressant drug, CPA, on the antiviral antibody response to VSV infections are investigated. CPA is an alkylating agent used as a chemotherapeutic drug against cancer, as an immunosuppressant for treating autoimmune diseases, for use in solid organ transplantations and in clinical trials with OVs. CPA suppresses the primary and anamnestic antiviral antibody response during oncolytic virotherapy with different viruses, including VSV. A prior study in mice investigated different treatment regimens and found that 4 days of CPA treatment (3 mg per dose) by the intraperitoneal (i.p.) route completely suppressed induction of antiviral antibodies against VSV challenge. Accordingly, the same CPA regimen in these studies is used Immunocompetent C57/b16 mice (n=8 per group) receive 4 daily doses of CPA (3 mg per mouse per dose, i.p.) beginning 1 day before VSV infection (106 pfu, i.p.) (FIG. 4A). At 28 days, mice are bled through the tail vein to determine anti-VSV antibody titers in the serum. Sera is heat treated to inactivate complement, diluted and incubated with live VSV prior to performing viral plaque assays on L929 indicator cells. Prior studies show that sunitinib affects the adaptive immune response. Although a study in cancer patients found that sunitinib did not impair seroprotection from influenza virus vaccination, it is unknown if sunitinib affects the antibody response to VSV as used in these studies. Therefore, we determine if daily doses of suntinib (20 mg/kg) by oral gavage beginning one day before inoculation with VSV affect the production of antiviral antibodies against VSV (FIG. 4B).

### Example 5

A syngeneic mouse model is used to evaluate effects of the adaptive immune response on OV therapy of prostate cancer. TRAMP-C1 cells, derived from prostate epithelium of TRAMP mice are tumorigenic when implanted into syngeneic C57/b16 mice. In preliminary studies, we showed that TRAMP-C1 cells express functional RNase L and PKR (FIGS. 1C-D). TRAMP-C1 cells (5×10⁶ cells in 0.1 ml PBS) are implanted s.c. into a single flank of C57/b16 mice (n=8 per group). When tumors reach 6-8 mm diameter (about 12 d), different groups of mice receive either sunitinib (oral gavage 20 mg/kg/d) prior to VSV (106 pfu, i.t.), or 4 daily doses of CPA (3 mg per mouse per dose, i.p.) beginning 2 days before VSV infection. These treatments are repeated after two weeks as indicated (FIG. 4C). Tumor growth is monitored daily throughout the course of the experiment. If tumors completely regress, we discontinue treatment and monitor mice for up to one month to determine if tumors return. Antiviral antibody titers are determined by collecting blood from the tail veins at 21 and 35 days. Titers of antibodies are measured by incubating heat-inactivated sera with VSV prior to performing viral plaque assays. The experiment with i.v. inoculations of VSV through the tail vein (FIG. 4C) is repeated to determine effects of CPA and sunitinib on antiviral antibody titers and oncolytic virotherapy in C56/b16 mice.

### Example 6

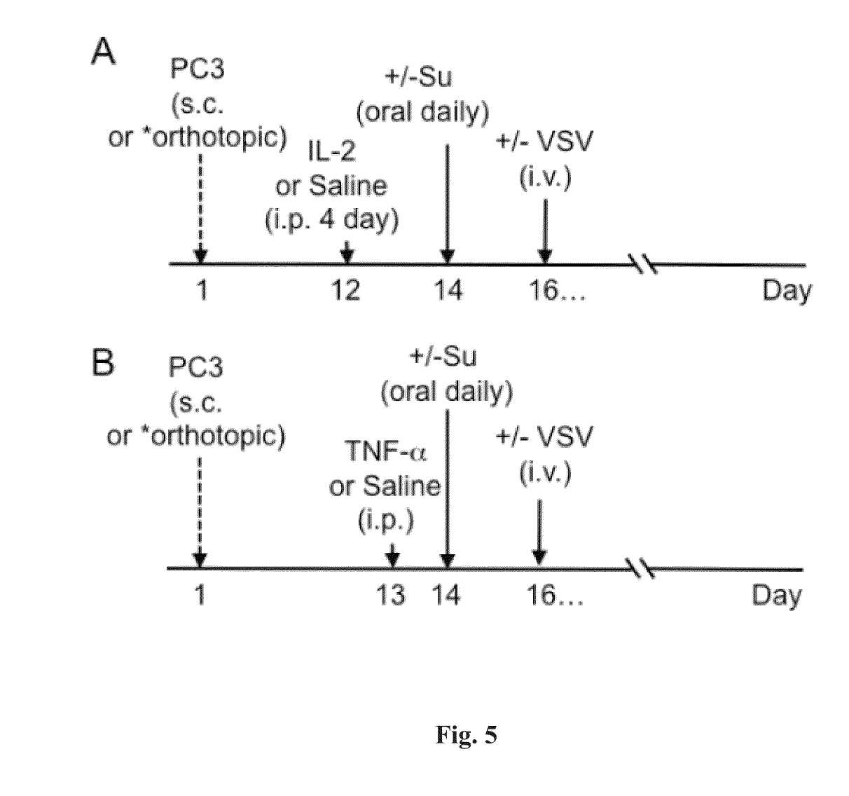

Extravasation of systemically delivered virus from blood vessels into tumor tissues is essential to treat metastatic prostate cancer. In these studies, viral titers in prostate tumor tissues following systemic delivery of virus is determined PC3 cells (2×10⁶ in matrigel) are implanted s.c. in nude mice. When tumors reach 6-8 mm diameter (about 12d), mice are treated with cytokines that enhance vascular permeability. IL-2 treatments cause endothelial cell injury and vascular leak syndrome (VLS) mediated by activated lymphocytes that bind and lyse endothelial cells. A prior study showed that non-toxic doses of IL-2 induced VLS and allowed intravenously delivered VSV to infiltrate metastatic lung tumors. A previously established regimen is used of 75,000 U/i.p. injection of recombinant human IL-2 (Cleveland Clinic pharmacy) is used, which is active in mice. IL-2, or as a control saline, is delivered three times a day for 3 days, followed by a single injection on the fourth day, one day prior to i.v. delivery of VSV (10⁸ pfu in 0.1 ml PBS) (FIG. 5A). Similarly, TNF-α was shown to enhance vascular permeability and adenovirus delivery to tumor tissues. A single i.p. injection of 1 μg recombinant TNF-α (R&D Systems) in 0.1 ml PBS per mouse is performed one day prior to i.v. inoculation with VSV to enhance vascular permeability and delivery of VSV to the tumor tissues (FIG. 5B). The effects are compared of IL-2 or TNF-α pre-treatments on virus titers in the tumors in the presence and absence of sunitinib treatments (delivered by oral gavage, 20 mg/kg/daily) beginning two days prior to infection. Mice are euthanized one week after VSV infections, tumors are excised, and viral titers measured in homogenates of the tumor tissues by viral plaque assays on L929 indicator cells.

### Example 7

As a mouse model in which both primary tumor growth and metastasis can be monitored in vivo, androgen-independent PC3 cells are implanted orthotopically in mice. PC3-luc cells, which stably express firefly luciferase are used. PC3-luc cells (2×10⁶ in matrigel) are inoculated into prostate of 5-6 week old male athymic nude homozygous (nu/nu) NCR mice (Taconic) (n=8 per group) as we described previously. Progression of primary tumors as well as metastasis is monitored twice a week using real-time in vivo imaging. Mice are anaesthetized and placed in a 37° C. light-tight chamber. Tumors are visualized, including distant micro-metastases, and volume determined by detection of emitted light using a IVIS 100 System (Xenogen). Mice are injected with luciferin (3 mg/mouse i.p.), anaesthetized (inhaled isoflurane), and imaged simultaneously at 10 min after luciferin injection. When the metastatic tumors are detectable by real-time imaging, daily treatments are started with sunitinib by oral gavage (20 mg/kg) or for comparison other mice are left untreated. After 2 days, mice are infected with VSV (10⁶ pfu) by the i.v. route, or left uninfected. Groups of mice are untreated (saline), sunitinib alone, VSV alone, and the combination of sunitinib and VSV (FIGS. 5A-B). The body weight of each animal is recorded daily.

Animal survival is monitored daily for euthanasia criteria indicating death is imminent, including obvious morbidity defined as at least two of the following four sequelae: general inactivity, difficult ambulating, ruffled fur and huddled posture. Following euthanasia, histology is performed on H&E stained FFPE sections of prostate, lymph nodes, bone and lungs (and other sites of metastasis) for evidence of primary tumors and metastasis. In separate experiments, the PC3/orthotopic model is used to determine the effects on animal survival of pre-treating with IL-2 or TNF-α to enhance extravasation prior to the initial VSV infection (FIGS. 5A-B). At the end of the experiments, mice are euthanized and sites of primary and metastatic tumors are subjected to histological examination.

### Example 8

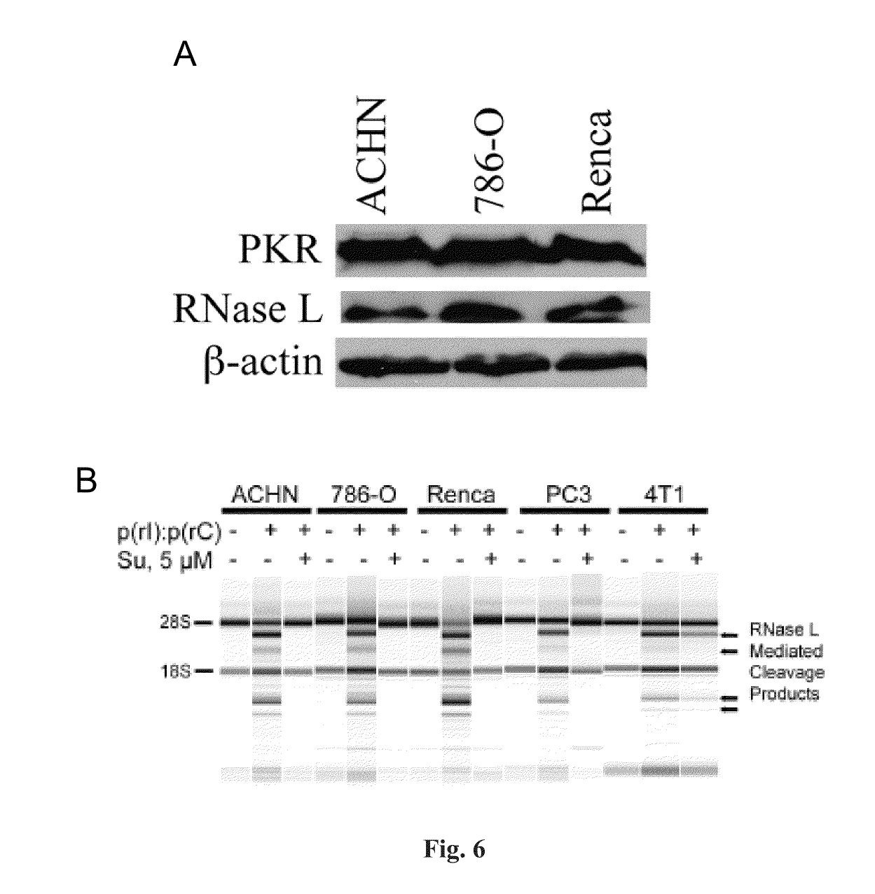

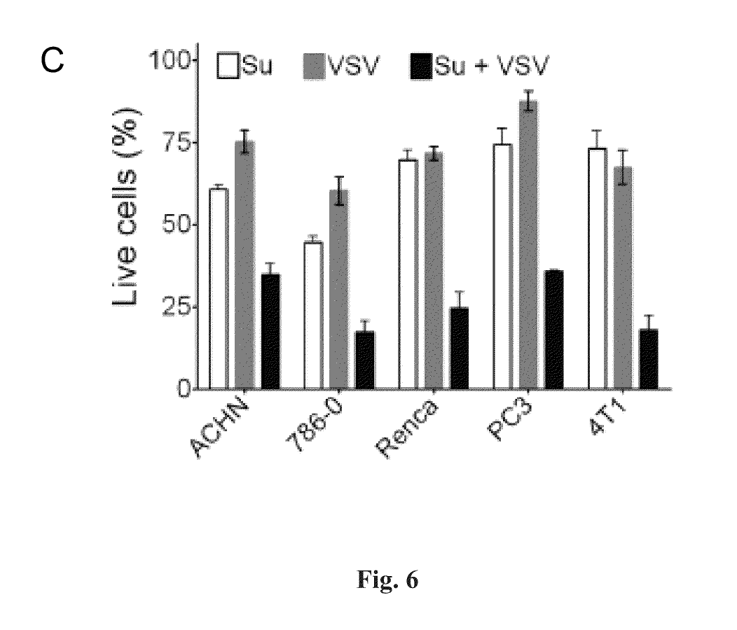

To determine the effect of sunitinib on RNase L activity in intact cancer cells, human kidney cancer (ACHN, 786-0, and Renca), human prostate cancer (PC3), and mouse breast cancer (4T1) cell lines were transfected with poly(rI):poly(rC) for 3 hrs to activate OAS and RNase L, total RNA was isolated and separated on RNA chips (FIG. 6B). Western blots probed with specific antibodies showed that kidney cancer cell lines, ACHN, 786-0, and Renca all express both PKR and RNase L (FIG. 6A). RNase L cleaves rRNA inintact ribosomes producing a unique and characteristic set of discrete RNA cleavage products. As expected, RNase L activity against rRNA was observed in the poly(rI):poly(rC)-transfected cancer cells, whereas there was little or no rRNA cleavage products in the cells treated with sunitinib prior to poly(rI):poly(rC)-transfection. Therefore, these cells have an intact OAS-RNase L pathway and sunitinib treatment effectively blocked RNase L activation in all of the cancer cell lines. Previously, we also showed that sunitinib effectively inhibits PKR activity in intact cells and in mice. The effect of sunitinib and VSV on survival of kidney cancer (ACHN, 786-0, Renca), prostate cancer (PC3), and breast cancer (4T1) cell lines was determined (FIG. 6C). Cells were pre-incubated in the absence or presence of sunitinib (5 μM) for 2 h and were either left uninfected or were infected with VSV at amultiplicity of infection (MOI) of 1.0 for 8 h (a single cycle of virus replication). Cell viability was measured by MTS assays using the CellTiter 96® Aqueous kit (Promega Inc.).

There was some reduction in cell viability by sunitnib or VSV alone. Interestingly, however, the combination of sunitinib and VSV greatly enhanced cell death in all of the kidney cancer cells, regardless of whether they were wild type (Renca, ACHN) or mutant (786-0) for VHL. Similarly, there was enhanced cell death in the PC3 and 4T1 cells by the combination treatment of sunitinib with VSV. These cell culture studies suggested that sunitinib renders cancer cells more susceptible to the lytic effect of VSV infections by impairing innate immunity, an effect that is independent of angiogenesis.

### Example 9

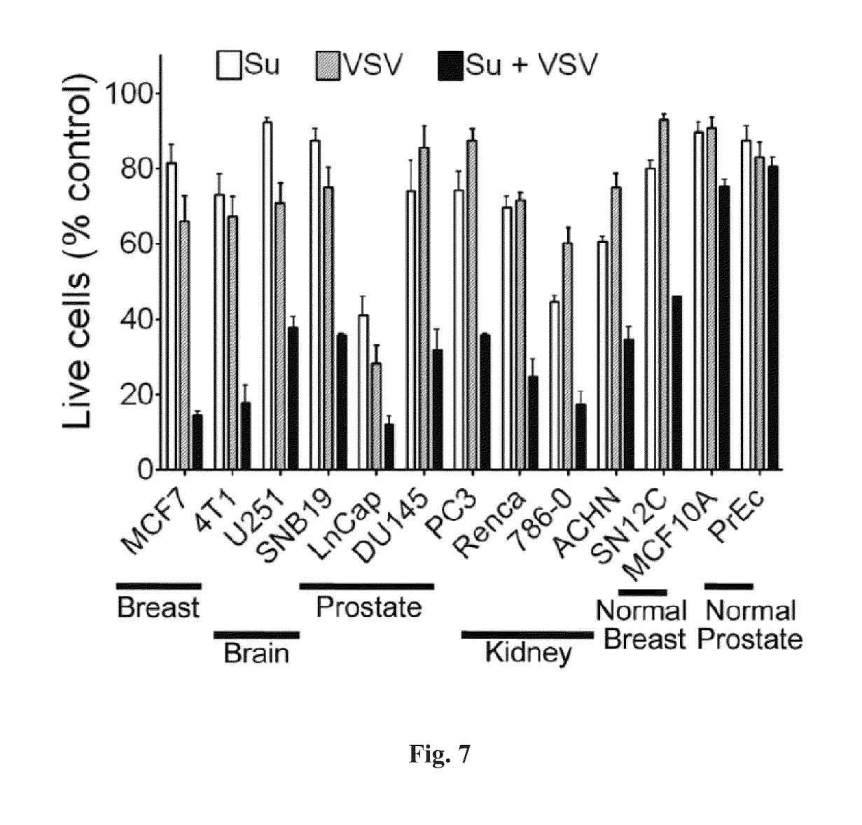

We determined the effect of sunitinib and VSV on a wide spectrum of different cancer cells (breast cancer cells MCF7 and 4T1; brain tumor cells U251 and SNB19; prostate cancer cells PC3, DU145 and LNCaP), renal cell carcinoma (Renca, ACHN, 786-O, SN12C and RCC4) as well as on the non-tumorigenic mammary epithelial cell line (MCF10A) and primary prostatic epithelial cells (PrEC) (FIG. 7). Cells were pre-incubated in the absence or presence of sunitinib (5 uM) for 2 h and were either left uninfected or were infected with VSV at a multiplicity of infection (MOI) of 1.0 for 8 h (a single cycle of virus replication). Cell viability was measured by MTS assays using the CellTiter 96® Aqueous kit (Promega Inc.). There was a minimal (typically about 20%) reduction in cell viability by sunitnib treatment alone, except for the LNCaP and 786-O cells in which suntinib reduced cell viability by 60%, perhaps indicating off-target effects in kinase(s) important for the survival of these cells. VSV infection alone reduced the viability of the cancer cells by <40% (except for LNCaP), while reducing viability of the MCF10 and PrEc cells by 15% and 20%, respectively. Interestingly, the combination of sunitinib and VSV greatly enhanced cell death in all of the cancer cells (60% to 90%), whereas the non-tumorigenic mammary epithelial cell line (MCF10A) and primary prostatic epithelial cells (PrEC) were resistant to the combination treatment (25% and 20% reduction in cell viability, respectively). These findings show that the combination treatment of VSV with sunitinib results in a broad-spectrum anti-tumor cell response while having minimal effects on normal cells Importantly, PC3 cells, which are normally resistant to VSV, presumably due to an active OAS/RNase L and PKR system, are rendered susceptible to VSV by sunitinib treatment

### Example 10

To determine effects of sunitinib on innate immunity pathways in vivo, activation of both RNase L and PKR are monitored in tumors implanted s.c. in mice. Clear-cell renal cell carcinoma (CCRCC) is the most common form of kidney cancer and also the most resistant to chemotherapy and radiation. Inactivation of the VHL tumor suppressor gene is responsible for most cases of CCRCC. Loss of VHL renders CCRCC cells more susceptible to OVs. Therefore, in these experiments we compare 786-O CCRCC VHL−/− cells either reconstituted with VHL (786-VHL) or expressing the empty plasmid (786-mock). Sunitinib is obtained from the Cleveland Clinic pharmacy and dissolved in PBS at 4 mg/ml. Cells (2×10⁶ in media plus matrigel, 1:1, in a final volume of 150 μl) are implanted s.c. into a single flank of athymic nude homozygous (nu/nu) NCR mice (Taconic, obtained through the Case Comprehensive Cancer Center, Cleveland). Tumor sizes are measured daily with a digital caliper. When the tumors reach 6-8 mm in diameter (typically 20-23 days), mice are divided into four groups (n=5): no treatment; sunitinib alone (20 mg/kg daily by oral gavage); VSV intratumoral (i.t.) inoculation alone [10⁶ plaque forming units (pfu)/mouse] two days later; and sunitinib (daily) followed 2 days later with VSV i.t. inoculation. At 24, 48 and 72 hrs postinfection mice are euthanized, blood collected, and the tumors excised. Tumors are immediately cut each into three parts with a scalpel and individual sections placed in dry ice, RNAlater (Qiagen), and formalin. Tumor tissue frozen in dry ice is thawed in PBS and homogenized using a Sample Grinding kit including a silica beads and a pestle (GE Healthcare). After centrifuging, supernatants are collected and used for performing viral plaque assays on the indicator mouse cell line, L929. Viral plaque assays are performed on sera collected at the time of euthanasia. The supernatants of the tissue homogenates are also used for Western blot assays. Total and phosphorylated levels of PKR and its substrate, eIF2α, are measured in Western blots by probing with specific antibodies (Cell Signaling, Inc.). RNase L activity is monitored in tumors by examining the integrity of rRNA in tumor samples preserved in RNA later. During viral infections, RNase L activation leads to the cleavage of both 285 and 18S at unique sites, as we showed previously (FIG. 1D). To identify RNase L mediated cleavage products in rRNA, the RNA is isolated using TRIzol (LifeTechnologies) and separated in RNA chips on an Agilent BioAnalyzer 2100. In addition, viral growth in tumors is assessed in fixed tissue sections. Formalin fixed and paraffin embedded (FFPE) tissues are sectioned and stained with antibody to VSV G protein (KeraFAST, Inc.).

### Example 11

To determine how sunitinib enhances oncolytic virotherapy in vivo, we establish the contributions of RNase L and PKR, individually and together, on tumor growth. Previously, it was reported that a vaccinia virus encoded protein, E3L, inhibits both RNase L and PKR by sequestering dsRNA, an activator of OAS (and subsequently RNase L) and PKR (FIG. 3). We stably express E3L in ACHN cells in order to simultaneous inhibit RNase L and PKR. The E3L cDNA (purchased as a synthetic cDNA from GenScript) is expressed as an N-terminal flag-tag protein in mammalian expression vector pCMV-ENTRY (Origene). ACHN cells are transfected with the E3L construct and selected for stable expression with G418. E3L expression is monitored in Western blots with antibody against the flag-tag. To demonstrate that E3L is functional, the ACHN cells expressing E3L, or containing empty vector as a control, are transfected with poly(rI):poly(rC) (1 μg/ml) for 3 hrs. RNase L activation is monitored by rRNA cleavages in RNA chips, whereas PKR activation is monitored in Western blots probed with antibody against phosphorylated and unphosphorylated PKR and eiF2α. ACHN/E3L and ACHN/control cells are then implanted into the flanks of nude mice and proceed with the tumor growth experiments as described (above) with four groups of mice (n=8): no treatment; VSV alone; sunitinib (daily) alone; and VSV plus sunitinib (daily). By comparing tumor growth rates between the vector control and E3L expressing ACHN cells, the contributions of RNase L and PKR (together) on tumor regression in response to VSV and sunitinib is established.

Recently, we reported that the ns2 gene of the coronavirus, MHV, encodes a protein that shuts down the OAS/RNase L pathway. Ns2 is a 2′,5′-phosphodiesterase that degrades 2-5A, the activator of RNase L, and thus prevents RNA cleavage by this enzyme. To selectively block the OAS-RNase L system, we stably express the ns2 protein from a cDNA in ACHN cells. We confirm ns2 expression in Western blots and then determine if RNase L activity is blocked by transfecting with poly(rI):poly(rC) prior to examining rRNA in RNA chips. Subsequently, the ACHN cells expressing MHV ns2 are implanted s.c. into nude mice and VSV/sunitinib treatments performed. By monitoring tumor growth, we determine the contribution of RNase L by itself to the antitumor effects of VSV in the presence and absence of sunitinib against ACHN tumors in mice.

To determine the relative contribution of both RNase L and PKR, we separately down-regulate RNase L and PKR levels in ACHN cells with lentiviruses encoding different shRNAs against mRNAs for RNase L or PKR. RNase L and PKR levels are monitored in Western Blots probed with a monoclonal antibody against human RNase L that we prepared, and antibody against PKR (from Cell Signaling, Inc.). Subsequently, the cells are transfected with poly(rI):poly(rC) prior to monitoring rRNA cleavage in RNA chips and phosphorylated and unphosphorylated PKR and eIF2α in Westernblots. We generate ACHN cells that stably express the most effective shRNA against either RNaseL or PKR. Effects of suntinib and VSV on cell survival are determined in ACHN cells in culture byMTS assays and then against s.c. implanted tumors in nude mice treated with sunitinib and VSV alone and in combination.

### Example 12

A syngeneic mouse model is used to evaluate effects of the adaptive immune response on OV therapy of kidney cancer. We investigate the effects the immunosuppressant drug, CPA, on the antiviral antibody response to VSV infections. CPA is an alkylating agent used as a chemotherapeutic drug against cancer, as an immunosuppressant for treating autoimmune diseases, for use in solid organ transplantations and in clinical trials with OVs. CPA suppresses the primary and anamnestic antiviral antibody response during oncolytic virotherapy with different viruses, including VSV. A prior study in mice investigated different treatment regimens and found that 4 days of CPA treatment (3 mg per dose) by the intraperitoneal (i.p.) route completely suppressed induction of antiviral antibodies against VSV challenge. Accordingly, we use the same CPA regimen in these studies. Mouse Renca cells (ATCC CRL-2947) (5×10⁶ cells in 0.1 ml PBS) are implanted s.c. into a single flank of syngeneic BALB/c mice (n=8 per group). When tumors reach 6-8 mm diameter (about 12 d), different groups of mice receive sunitinib (oral gavage 20 mg/kg/d) prior to VSV (10⁶ pfu, i.t.), or 4 daily doses of CPA (3 mg per mouse per dose, i.p.), or saline (control) beginning 2 days before VSV infection. These treatments are repeated after two weeks as indicated (FIG. 4D). Tumor growth is monitored daily throughout the course of the experiment. If tumors completely regress, we discontinue treatment and monitor mice for up to one month to determine if tumors return. Antiviral antibody titers are determined by collecting blood from the tail veins at 21 and 35 days. Titers of antibodies are measured by incubating heat-inactivated sera with VSV prior to performing viral plaque assays. To determine effects of CPA and sunitinib on the ability of VSV to transit blood vessels and enter tumor tissues, we repeat the experiment with i.v. inoculations of VSV through the tail vein (FIG. 4D).

### Example 13

ACHN cells that stably express the firefly luciferase cDNA (ACHN-luc) (GenScript) are used for real-time in vivo imaging. ACHN cells produce metastasis in mice when implanted orthotopically in the subrenal capsule. Therefore, as a mouse model in which both primary tumor growth and metastasis can be monitored in vivo, we implant ACHN-luc (10⁵ cells) under general anaesthetic, into the renal capsule of athymic nude mouse (n=8/group). Progression of primary tumors as well as metastasis is monitored twice a week using real-time in vivo imaging. Mice are anaesthetized and placed in a 37° C. light-tight chamber. Tumors are visualized, including distant micro-metastases, and volume determined by detection of emitted light using a IVIS 100 System (Xenogen). Mice are injected with luciferin (3 mg/mouse i.p.), anaesthetized (inhaled isoflurane), and imaged simultaneously at 10 min after luciferin injection. When the metastatic tumors are detectable by real-time imaging we begin daily treatments with sunitinib by oral gavage (20 mg/kg) or for comparison other mice are left untreated. After 2 days, mice are infected with VSV (10⁶ pfu) by the i.v. route, or left uninfected. In addition, we first infect with VSV i.v. to allow virus to be taken up by leaky vessels in tumors, followed 2 days later with the initial sunitinib treatment (by oral gavage daily). This protocol inhibits RNase L and PKR thereby allowing spread of the virus throughout the tumors.

The body weights of the animals are recorded daily. Animal survival is also monitored daily for euthanasia criteria indicating death is imminent, including obvious morbidity defined as at least two of the following four sequelae: general inactivity, difficult ambulating, ruffled fur and huddled posture. Following euthanasia, histology is performed on H&E stained FFPE sections of kidney, lymph nodes, bone and lungs (and other sites of metastasis) for evidence of primary tumors and metastasis.

From the above description, those skilled in the art will perceive improvements, changes and modifications. Such improvements, changes, and modifications are within the skill of those in the art and are intended to be covered by the appended claims. All patents, patent applications, and publications cited herein are incorporated by reference in their entirety.

1. A method for treating a subject with cancer, the method comprising administering an oncolytic virus simultaneously, sequentially, or separately in combination with an immunomodulatory agent in an amount effective to suppress both antiviral immunity and angiogenesis associated with the cancer.

2. The method of claim 1, wherein the cancer is prostate cancer, breast cancer or renal cancer.

3. The method of claim 2, wherein the prostate cancer is castration-resistant prostate cancer.

4. The method of claim 1, wherein the cancer is a solid tumor.

5. The method of claim 1, wherein the oncolytic virus is a native or modified rhabdovirus.

6. The method of claim 5, wherein the oncolytic virus is a native or modified vesicular stomatitis virus.

7. The method of claim 1, wherein the immunomodulatory agent:
suppresses angiogenesis associated with the cancer; and
suppresses innate immunity, adaptive immunity, or a combination thereof, to the oncolytic virus.

8. The method of claim 7, wherein the immunomodulatory agent is a kinase inhibitor of vascular endothelial growth factor receptor and platelet-derived growth factor receptor.

9. The method of claim 7, wherein the immunomodulatory agent suppresses the activity of RNase L and RNA-dependent protein kinase.

10. The method of claim 7, wherein the immunomodulatory agent is sunitinib.

11. The method of claim 1, wherein the immunomodulatory agent is an alkylating agent capable of suppressing adaptive immunity to the oncolytic virus.

12. The method of claim 11, wherein the alkylating agent is cyclophosphamide.

13. The method of claim 1, further comprising the step of administering a cytokine to the subject prior to administration of the oncolytic virus and the immunoregulatory agent in an amount effective to promote vascular permeability and enhance delivery of the oncolytic virus to a cancer cell.

14. A method for treating or ameliorating a solid tumor in a subject, the method comprising:
(a) administering to the subject an effective amount of an oncolytic virus to the solid tumor, the oncolytic virus being capable of selectively killing cells of the solid tumor; and
(b) administering to the subject an immunomodulatory agent in an amount effective to suppress both angiogenesis associated with the solid tumor and innate immunity to the oncolytic virus.

15. The method of claim 14, wherein the solid tumor is prostate cancer, breast cancer or renal cancer.

16. The method of claim 14, wherein step (a) is performed before step (b).

17. The method of claim 14, wherein step (a) is performed after step (b).

18. The method of claim 14, wherein step (a) is performed simultaneous with step (b).

19. The method of claim 14, wherein the immunomodulatory agent is sunitinib.

20. A method for modulating sensitivity of a cancer cell to infection by an oncolytic virus, the method comprising:
(a) contacting the cancer cell with the oncolytic virus; and
(b) introducing into the subject an immunomodulatory agent in an amount effective to modulate antiviral immunity and sensitize the cancer cell to cytolytic activity of the oncolytic virus.

21. The method of claim 20, wherein the immunomodulatory agent:
suppresses angiogenesis associated with the cancer cell; and
suppresses innate immunity, adaptive immunity, or a combination thereof, to the oncolytic virus.

22. The method of claim 21, wherein the immunomodulatory agent is sunitinib.
